(19) United States
(12) Patent Application Publication
(10) Pub. No.: US 2025/0387594 A1
(43) Pub. Date: Dec. 25, 2025

(54) INTERMITTENT URINARY CATHETER STRUCTURE

(71) Applicant: GUANGZHOU WELLLEAD MEDICAL CO., LTD., Guangzhou (CN)

(72) Inventors: Yangping XIAO, Guangzhou (CN); Dong CAO, Guangzhou (CN); Guangyuan HAN, Guangzhou (CN); Bin XIANG, Guangzhou (CN)

(21) Appl. No.: 19/306,463

(22) Filed: Aug. 21, 2025

Related U.S. Application Data
(63) Continuation of application No. PCT/CN2023/124645, filed on Oct. 16, 2023.

(30) Foreign Application Priority Data
Feb. 24, 2023 (CN) 202310161411.8
Feb. 24, 2023 (CN) 202320305959.0

(51) Int. Cl. A61M 25/00; A61M 39/10; A61M 39/22

(52) U.S. Cl. CPC A61M 25/0045; A61M 39/10; A61M 39/22; A61M 2025/0006; A61M 2025/0046; A61M 2039/1027; A61M 2039/1033; A61M 2205/0216; A61M 2205/0222; A61M 2205/0238; A61M 2205/3331; A61M 2210/1089

(57) ABSTRACT

Disclosed is an intermittent urinary catheter structure, which comprises: a tube, one end of the tube acting in concert with the human body and the other end of the tube being provided with a switch used for controlling the starting and stopping of liquid flow, and the outside of the tube having sleeved thereon a retractable sheath extending along the length direction of the tube, one end of the sheath being connected to the switch, and the other end of the sheath being detachably provided with a sealing cap, forming a closed structure capable of enveloping the tube. The present application has the advantages of reducing the risk of infection and the difficulty of tube insertion.

### CROSS-REFERENCE TO RELATED APPLICATIONS

This application is a Continuation of co-pending International Application No. PCT/CN2023/124645, filed on Oct. 16, 2023, for which priority is claimed under 35U.S.C. § 120; and this application claims priority of Application No. 202310161411.8 filed in China on Feb. 24, 2023 and Application No. 202320305959.0 filed in China on Feb. 24, 2023 under 35 U.S.C. § 119; the entire contents of all of which are hereby incorporated by reference.

### TECHNICAL FIELD

The present disclosure relates to the field of medical devices, and in particular to an intermittent urinary catheter structure.

### BACKGROUND

Intermittent urinary catheters are usually used for patients with bladder functions being out of control due to diseases or other factors such as spinal cord injury, for example, multiple sclerosis or spina bifida, or patients needed to urinate temporarily after urethral surgery, bladder surgery, ureteral surgery or urinary diversion surgery and patients suffering from chronic urinary retention needed to urinate in daily life. The patients themselves or nursing staff insert and pull out the intermittent urinary catheters 4-6 times every day to intermittently drain urine from the bladders of the patients. During emptying every time, the intermittent urinary catheters stay in the urethras 10-15 minutes or shorter. Compared with indwelling urinary catheterization that has to be retained in the urethras several weeks, intermittent catheterization can stimulate recovery of bladder functions and effectively reduce the risk of urinary tract infection. However, for an existing common intermittent urinary catheter, particularly a male intermittent urinary catheter, due to a long tube, in the process of inserting the urinary catheter into the urethra, it is difficult to prevent a hand of an operator from contacting the tube of the urinary catheter. Such a contact directly damages the sterility of the tube, which increases the risk of infection since bacteria enter the urethra.

In order to reduce the risk of urinary tract infection, Chinese Patent Application No. CN205307468U discloses a portable intermittent type catheterization combination package. The urinary catheter is put in a urinary catheter bag. During urethral catheterization, the operator picks up the urinary catheter bag and tears off a head packaging and gradually retreats the urinary catheter bag after the urinary catheter is inserted into the urethra, so that the hand of the operator is prevented from directly contacting the urinary catheter, thereby preventing the risk of infection to the maximum extent. However, in order to save cost, most patients will clear and disinfect the urinary catheter and put the urinary catheter in a special sheath after emptying urine, so that the urinary catheter may be repeatedly used. That is, the urinary catheter is put in the urinary catheter bag, which is a disposable article. The urinary catheter bag plays a protective role only when the urinary catheter is used first time. When the patient repeatedly uses the urinary catheter subsequently, the hand of the operator cannot be prevented from directly contacting the urinary catheter through the urinary catheter bag continuously, so that the risk of infection is increased since bacteria enter the urethra. In addition, the urinary catheter is an once-through tube simple in structure. During intermittent urinary catheterization, it usually needs to pinch a connector of the urinary catheter or bend part of the urinary catheter to avoid a situation that at a moment when the urinary catheter is put in the bladder, the urine spouts out and splashes outside to contaminate the hand of the operator, clothing, environment, and the like. This operation makes the operator insert the tube only with a single hand to a certain extent, so that the difficulty of tube insertion is increased.

How to solve the above problem becomes a technical problem needed to be solved urgently.

### SUMMARY

An objective of the present disclosure is to provide an intermittent urinary catheter structure capable of reducing the risk of infection and decreasing the difficulty of tube insertion.

In order to achieve the above objective, the present disclosure adopts the following technical solutions:

An intermittent urinary catheter structure includes a tube, where one end of the tube acts in concert with a human body and the other end of the tube is provided with a switch used for controlling the starting and stopping of liquid flow, an outside of the tube has sleeved thereon a retractable sheath extending along a length direction of the tube, one end of the sheath is connected to the switch and the other end of the sheath is detachably provided with a sealing cap, forming a closed structure capable of enveloping the tube.

Further, the switch includes a switch body, an end of each of the tube and the sheath is fixedly connected to the switch body, the switch body is internally provided with a drainage channel, one end of the drainage channel is in fluid communication with the tube and the other end of the drainage channel is in fluid communication with the outside through a drainage chamber, an opening is formed in a top of the switch body, an L-shaped guide post is slidably arranged in the opening, one end of the guide post is located in the drainage chamber and is in plug-in fit with the drainage channel to form a structure capable of blocking the drainage channel, the other end of the guide post extends out of the drainage chamber via the opening and is connected to a push cover capable of covering the opening, a leak-proof ring enveloping the opening is arranged on a top surface of the switch body, and the leak-proof ring fits a bottom surface of the push cover closely to form a sealing structure capable of preventing liquid from being leaked from the opening.

Further, snap fasteners are symmetrically arranged on both sides of the push cover, guide slots extending in a length direction of the switch body are symmetrically formed in side walls of both sides of the switch body, and the guide slots are in sliding fit with the snap fasteners.

Further, an end of the guide post distal to the push cover is provided with a blocking ring capable of blocking the drainage channel.

Further, the switch includes a rotary valve, the rotary valve is in fluid communication with the tube through a connector, and an end of each of the tube and the sheath distal to the sealing cap is fixedly connected to an end of the connector distal to the rotary valve.

Further, the rotary valve includes a valve body penetrated at two ends, an end of the connector distal to the tube is in fluid communication with the valve body, the valve body is internally provided with a valve cavity, a valve stem is rotationally arranged in the valve cavity, the valve stem is in transition fit with the valve cavity and the valve stem is provided with a valve hole radially penetrating the valve stem, and an upper end of the valve stem extends outside the valve body and is connected to a valve handle.

Further, an end of the sheath distal to the switch is provided with an annular guide connector, one end of the guide connector is in fluid communication with the sheath, and the other end of the guide connector is detachably connected to the sealing cap.

Further, one end of the tube distal to the switch is provided with a drainage hole, the other end of the tube is provided with a pressure relief hole, and the tube is in fluid communication with the sheath through the pressure relief hole.

Further, an outer surface of the tube is coated with a lubricating coating.

Further, the sheath is of a flexible cylindrical film structure.

With the adoption of the above structure, the present disclosure has the following beneficial effects:

By arranging the switch, when the end of the tube acting in concert with the human body is placed in the bladder and the urine flows out along the tube, the switch can prevent the urine from being sprayed outside instantaneously, and the operator can control the chance when the urine flows outside through the switch after successful tube insertion and does not need to pinch the tube or bent part of the tube during tube insertion, such that the difficulty of tube insertion is decreased; moreover, by arranging the sheath, in use, the sealing cap is opened and the end of the sheath proximal to the switch is retracted, such that the tube enveloped by the sheath can extend out of the sheath and can be put in the urethra, and after the tube is pulled out from the urethra, the sheath is stretched, such that the sheath envelops the tube again and is lidded with the sealing cap; therefore, during tube inserting and extubating operations, the hand pinches the tube across the sheath and is prevented from directly contacting with the tube, such that zero-contact operation is achieved, the contamination of bacteria on the tube is avoided, and the risk of urinary tract infection is reduced.

The present disclosure will become clearer through the description below in conjunction with the drawings. These drawings are used to explain embodiments of the present disclosure.

### BRIEF DESCRIPTION OF THE DRAWINGS

To describe the technical solutions in embodiments of the present disclosure or in the prior art more clearly, the drawings required for describing the embodiments or prior art are briefly described below. Apparently, the accompanying drawings in the following description show some embodiments of the present disclosure, and those of ordinary skill in the art may still derive other drawings from these drawings without making creative efforts.

FIG. 1 is a schematic diagram of an overall structure of embodiment I of the present disclosure.

FIG. 2 is a cross-sectional view of Embodiment I of the present disclosure.

FIG. 3 is a schematic diagram I of a part of structure of a switch in Embodiment I of the present disclosure.

FIG. 4 is a schematic diagram II of a part of structure of a switch in Embodiment I of the present disclosure.

FIG. 5 is a schematic structural diagram of Embodiment II of the present disclosure.

FIG. 6 is a cross-sectional view of Embodiment II of the present disclosure.

### DETAILED DESCRIPTIONS OF THE EMBODIMENTS

The technical solutions in the embodiments of the present disclosure will be described clearly and intactly below in conjunction with the accompanying drawings in the present disclosure. Apparently, the described embodiments are merely a part of the embodiments, rather than all the embodiments, of the present disclosure. Based on the embodiments in the present disclosure, all other embodiments obtained by those of ordinary skill in the art without creative labor all fall within the protection scope of the present disclosure.

Embodiment I

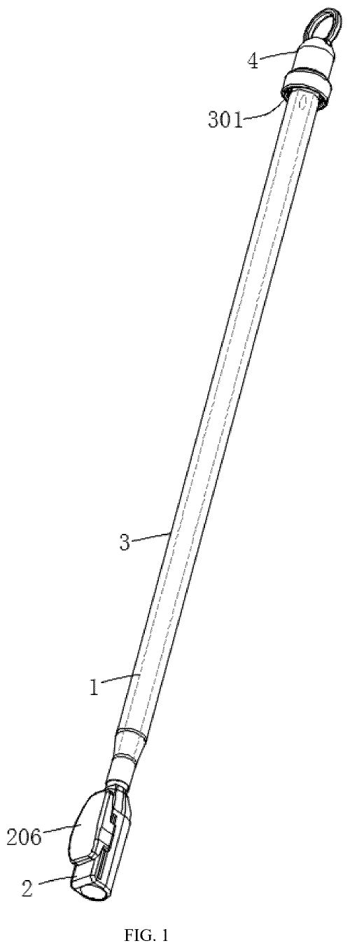

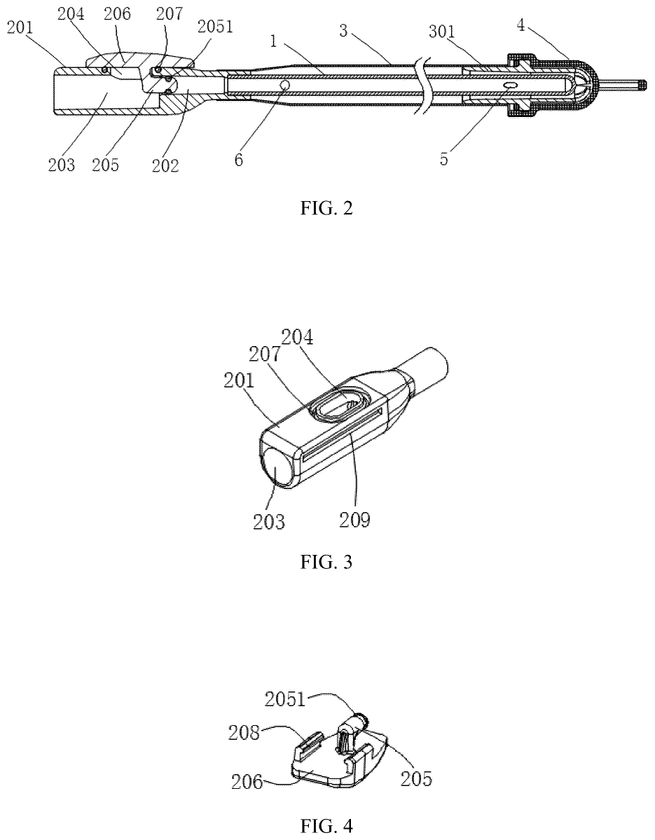

Referring to FIGS. 1-4, the intermittent urinary catheter structure provided by the present disclosure includes a tube 1, where one end of the tube 1 acts in concert with a human body and the other end of the tube 1 is provided with a switch 2 used for controlling the starting and stopping of liquid flow, such that it is not needed to pinch the tube 1 or bend part of the tube 1 during tube insertion, and therefore, the difficulty of tube insertion is decreased; an outside of the tube has sleeved thereon a retractable sheath 3 extending along the length direction of the tube 1, one end of the sheath 3 is connected to the switch 2 and the other end of the sheath 3 is detachably provided with a sealing cap 4, forming a closed structure capable of enveloping the tube 1. In use, the sealing cap 4 is opened and an end of the sheath 3 proximal to the switch 2 is retracted, such that an end, distal to the switch 2, of the tube 1 enveloped by the sheath 3 is exposed out of the sheath 3, an operator pinches the tube 1 across the sheath 3 and puts the tube into the urethra. Moreover, after urinary tube insertion, the tube 1 is pulled out from the urethra, and the sheath 3 is stretched, such that the sheath envelops the tube 1 again and is lidded with the sealing cap 4; and therefore, during urinary tube insertion, the tube 1 is prevented from being contaminated by the hand.

In this embodiment, the switch 2 includes a switch body 201, an end of each of the tube 1 and the sheath 3 distal to the sealing cap 4 is fixedly connected to the switch body 201, the switch body 201 is internally provided with a drainage channel 202, one end of the drainage channel 202 is in fluid communication with the tube 1 and the other end of the drainage channel 202 is in fluid communication with the outside through a drainage chamber 203, and moreover, an opening 204 is formed in a top of the switch body 201, an L-shaped guide post 205 is slidably arranged in the opening 204, one end of the guide post 205 is located in the drainage chamber 203 and is in plug-in fit with the drainage channel 202 to form a structure capable of blocking the drainage channel 202, the other end of the guide post 205 extends out of the drainage chamber 203 via the opening 204 and is connected to a push cover 206 capable of covering the opening 204, a leak-proof ring 207 enveloping the opening 204 is arranged on a top surface of the switch body 201, and the leak-proof ring 207 is made of a rubber or silica gel material; and the leak-proof ring 207 fits a bottom surface of the push cover 206 closely to form a sealing structure capable of preventing liquid from being leaked from the opening 204. In use, the push cover 206 is pulled back to drive the guide post 205 to move toward a direction distal to the tube 1 until the front end of the guide post 205 is separated from the drainage channel 202, so as to open the switch 2. The drainage chamber 203 is in fluid communication with the drainage channel 202, such that urine blocked in the tube 1 is discharged to the outside. The push cover 206 is pushed forward to drive the guide post 205 to move toward a direction proximal to the tube 1 until the front end of the guide post 205 blocks the drainage channel 202, so as to switch off the switch 2. The drainage chamber 203 is not in fluid communication with the drainage channel 202 to prevent the urine in the tube 1 from being drained to the outside.

In this embodiment, snap fasteners 208 are symmetrically arranged on both sides of the push cover 206, guide slots 209 extending in a length direction of the switch body 201 are symmetrically formed in side walls of both sides of the switch body, and the guide slots 209 are in sliding fit with the snap fasteners 208. The push cover 206 buckles the guide slots 209 through the snap fasteners 208 to limit movement of the push cover 206 in the vertical direction, such that the bottom surface of the push cover 206 clings to the leak-proof ring 207 to prevent liquid from being leaked from a gap between the opening 204 and the push cover 206.

In this embodiment, an end of the guide post 205 distal to the push cover 206 is provided with a blocking ring 2051 capable of blocking the drainage channel 202. The blocking ring 2051 is in zero clearance fit with the drainage channel 202, may also be made of rubber or a silica gel or another material capable of deforming elastically, and the blocking ring 2051 is sleeved on the outer wall of the guide post 205, such that when an end of the guide post 205 distal to the push cover 206 is inserted into the drainage channel 202, the blocking ring 2051 can block the drainage channel 202 to prevent the urine in the tube 1 from being leaked into the drainage chamber 203 via the gap between the guide post 205 and the drainage channel 202.

In this embodiment, an end of the sheath 3 distal to the switch 2 is provided with an annular guide connector 301, one end of the guide connector 301 is in fluid communication with the sheath 3, and the other end of the guide connector 301 is detachably connected to the sealing cap 4. The guide connector 301 is in screw thread fit or snap fit, or plug-in fit with the sealing cap 4.

In this embodiment, one end of the tube 1 distal to the switch is provided with a drainage hole 5, and the urine flows into the tube 1 via the drainage hole 5; the other end of the tube 1 is provided with a pressure relief hole 6, and the tube 1 is in fluid communication with the sheath 3 through the pressure relief hole 6, such that the gas and liquid in the tube 1 can be discharged into the sheath 3 to reduce the pressure formed by integrally closing the inner cavity. Before the switch 2 is switched on, the urine may enter the tube 1 under the pressure in the bladder.

In this embodiment, an outer surface of the tube 1 is coated with a lubricating coating. The lubricating coating is a semi-liquid gel or a polyester super lubricous coating adhered to the surface of the tube 1, and is configured to reduce the frictional force when the tube 1 passes through the urethra. In addition, the lubricating coating may further be configured to store a liquid lubricant or a coating liquid between the sheath 3 and the tube 1 to further reduce the frictional force during tube insertion.

In this embodiment, the sheath 3 is of a flexible cylindrical film structure, and the sheath 3 is made of PU, or PE, or another elastic material.

In use of this embodiment, first, the sealing cap 4 is removed from the guide connector 301, and then the end of the sheath 3 proximal to the switch 2 is retracted, such that the end, distal to the switch 2, of the tube 1 enveloped by the sheath 3 is exposed out of the sheath 3, the operator pinches the tube 1 across the sheath 3 and put the tube 1 into the urethra. The urine in the body of the patient can flow into the tube 1 via the drainage hole 5, then the end of the switch 2 distal to the tube 1 is aligned with a urine bucket or a urine bag or another container, the push cover 206 is pushed back to drive the guide post 205 to move toward the direction distal to the tube 1 until the front end of the guide post 205 is separated from the drainage channel 202, so as to switch on the switch 2, such that the drainage chamber 203 is in fluid communication with the drainage channel 202, and the urine blocked in the tube 1 is discharged to the outside; when the urine in the body of the patient is emptied, the push cover 206 is pushed forward to drive the guide post 205 to move toward the direction proximal to the tube 1 until the blocking ring 2051 blocks the drainage channel 202, so as to switch off the switch 2. Finally, the tube 1 is pulled out from the urethra, and after disinfection, the sheath 3 is stretched, such that the sheath envelops the tube 1 again and is lidded with the sealing cap 4; therefore, during urinary tube insertion, the tube 1 is prevented from being contaminated by the hand, and the sheath 3 can be repeated retracted and stretched to prevent the tube 1 from being contaminated by the hand of the operator many times.

### Embodiment II

This embodiment differs from embodiment I in that:

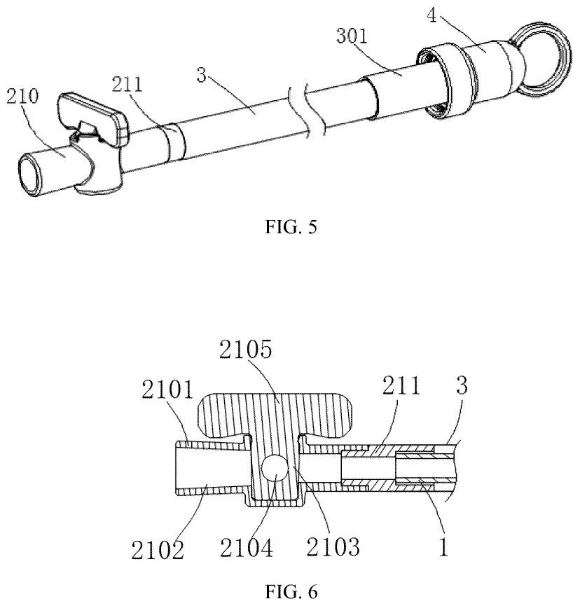

Referring to FIG. 5 and FIG. 6, in this embodiment, the switch 2 includes a rotary valve 210, the rotary valve 210 is in fluid communication with the tube 1 through a connector 211, and an end of each of the tube 1 and the sheath 3 distal to the sealing cap 4 is fixedly connected to an end of the connector 211 distal to the rotary valve 210. When the rotary valve 210 is opened, the urine can be drained to the outside through the tube 1, the connector 211, and the rotary valve 210 in sequence.

In this embodiment, the rotary valve 210 includes a valve body 2101 penetrated at two ends, an end of the connector 211 distal to the tube 1 is in fluid communication with the valve body 2101, the valve body 2101 is internally provided with a valve cavity 2102, a valve stem 2103 is rotationally arranged in the valve cavity 2102, the valve stem 2103 is in transition fit with the valve cavity 2102 and the valve stem 2103 is provided with a valve hole 2104 radially penetrating the valve stem 2103, and an upper end of the valve stem 2103 extends outside the valve body 2101 and is connected to a valve handle 2105. When an axis of the valve hole 2104 is perpendicular to an axis of the valve body 2101, the rotary valve 210 is closed to block the urine from flowing to the outside; when the valve stem 2103 is rotated through the valve handle 2105 to make both ends of the valve body 2101 be in fluid communication through the valve hole 2104, the rotary valve 210 is opened, and the urine can be discharged to the outside through the tube 1, the connector 211, and the rotary valve 210 in sequence. Moreover, the flow of the discharged urine may be adjusted by adjusting the included angle between the axis of the valve hole 2104 and the axis of the valve body 2101.

The above describes preferred embodiments of the present disclosure. It should be understood that the present disclosure is not limited to the above specific implementations, and devices and structures that are not described in detail should be understood as being implemented in common modes in the art; those skilled in the art may make various possible alternations and modifications on the technical solution of the present disclosure by means of the disclosed method and technical content without departing from the scope of the technical solution of the present disclosure or modify the technical solution into equivalently changed equivalent embodiments, which does not affect the essential content of the present disclosure. Therefore, any simple revisions, equivalent changes, and modifications made on the above embodiments according to the technical essence of the present disclosure without departing from the content of the technical solution of the present disclosure all fall within the protection scope of the technical solution of the present disclosure.

1. An intermittent urinary catheter structure, comprising a tube, wherein one end of the tube acts in concert with a human body and the other end of the tube is provided with a switch used for controlling the starting and stopping of liquid flow, an outside of the tube has sleeved thereon a retractable sheath extending along a length direction of the tube, one end of the sheath is connected to the switch, and the other end of the sheath is detachably provided with a sealing cap, forming a closed structure capable of enveloping the tube.

2. The intermittent urinary catheter structure according to claim 1, wherein the switch comprises a switch body; an end of each of the tube and the sheath distal to the sealing cap is fixedly connected to the switch body, the switch body is internally provided with a drainage channel, one end of the drainage channel is in fluid communication with the tube and the other end of the drainage channel is in fluid communication with the outside through a drainage chamber, an opening is formed in a top of the switch body, an L-shaped guide post is slidably arranged in the opening, one end of the guide post is located in the drainage chamber and is in plug-in fit with the drainage channel, forming a structure capable of blocking the drainage channel, the other end of the guide post extends out of the drainage chamber via the opening and is connected to a push cover capable of covering the opening, a leak-proof ring enveloping the opening is arranged on a top surface of the switch body, and the leak-proof ring fits a bottom surface of the push cover closely to form a sealing structure capable of preventing liquid from being leaked from the opening.

3. The intermittent urinary catheter structure according to claim 2, wherein snap fasteners are symmetrically arranged on both sides of the push cover, guide slots extending in a length direction of the switch body are symmetrically formed in side walls of both sides of the switch body, and the guide slots are in sliding fit with the snap fasteners.

4. The intermittent urinary catheter structure according to claim 2, wherein an end of the guide post distal to the push cover is provided with a blocking ring capable of blocking the drainage channel.

5. The intermittent urinary catheter structure according to claim 1, wherein the switch comprises a rotary valve, the rotary valve is in fluid communication with the tube through a connector, and an end of each of the tube and the sheath distal to the sealing cap is fixedly connected to an end of the connector distal to the rotary valve.

6. The intermittent urinary catheter structure according to claim 5, wherein the rotary valve comprises a valve body hollow and open at both ends, an end of the connector distal to the tube is in fluid communication with the valve body, the valve body is internally provided with a valve cavity, a valve stem is rotationally arranged in the valve cavity, the valve stem is in transition fit with the valve cavity and the valve stem is provided with a valve hole radially penetrating the valve stem, and an upper end of the valve stem extends outside the valve body and is connected to a valve handle.

7. The intermittent urinary catheter structure according to claim 1, wherein an end of the sheath distal to the switch is provided with an annular guide connector, one end of the guide connector is in fluid communication with the sheath, and the other end of the guide connector is detachably connected to the sealing cap.

8. The intermittent urinary catheter structure according to claim 1, wherein one end of the tube distal to the switch is provided with a drainage hole, the other end of the tube is provided with a pressure relief hole, and the tube is in fluid communication with the sheath through the pressure relief hole.

9. The intermittent urinary catheter structure according to claim 1, wherein an outer surface of the tube is coated with a lubricating coating.

10. The intermittent urinary catheter structure according to claim 1, wherein the sheath is of a flexible cylindrical film structure.

11. The intermittent urinary catheter structure according to claim 2, wherein an end of the sheath distal to the switch is provided with an annular guide connector, one end of the guide connector is in fluid communication with the sheath, and the other end of the guide connector is detachably connected to the sealing cap.

12. The intermittent urinary catheter structure according to claim 5, wherein an end of the sheath distal to the switch is provided with an annular guide connector, one end of the guide connector is in fluid communication with the sheath, and the other end of the guide connector is detachably connected to the sealing cap.

13. The intermittent urinary catheter structure according to claim 2, wherein one end of the tube distal to the switch is provided with a drainage hole, the other end of the tube is provided with a pressure relief hole, and the tube is in fluid communication with the sheath through the pressure relief hole.

14. The intermittent urinary catheter structure according to claim 5, wherein one end of the tube distal to the switch is provided with a drainage hole, the other end of the tube is provided with a pressure relief hole, and the tube is in fluid communication with the sheath through the pressure relief hole.

15. The intermittent urinary catheter structure according to claim 2, wherein an outer surface of the tube is coated with a lubricating coating.

16. The intermittent urinary catheter structure according to claim 5, wherein an outer surface of the tube is coated with a lubricating coating.

17. The intermittent urinary catheter structure according to claim 2, wherein the sheath is of a flexible cylindrical film structure.

18. The intermittent urinary catheter structure according to claim 5, wherein the sheath is of a flexible cylindrical film structure.
